(19) United States
(12) Patent Application Publication
(10) Pub. No.: US 2019/0328610 A1
(43) Pub. Date: Oct. 31, 2019

(54) SYSTEM AND METHOD FOR INTERACTIVE INTERVENTION BASED ON BIOMECHANICAL WAVE

(71) Applicant: Wisdom Qiao, Hacienda Heights, CA (US)

(72) Inventor: Wisdom Qiao, Hacienda Heights, CA (US)

(21) Appl. No.: 16/111,594

(22) Filed: Aug. 24, 2018

(30) Foreign Application Priority Data
Apr. 28, 2018 (CN) 201810405066.7

(51) Int. Cl. A61H 23/02; G16H 20/30; G16H 40/63

(52) U.S. Cl. CPC A61H 23/0236; G16H 20/30; G16H 40/63; A61H 2201/501; A61H 2201/1652; A61H 2201/163; A61H 2205/081; A61H 2201/5043; A61H 2201/1626

(57) ABSTRACT

The present application discloses a system for interactive intervention based on biomechanical wave. The system for interactive intervention based on biomechanical wave, comprising a biomechanical wave control system, a data computing device and a server. The biomechanical wave control system comprises a communication device, a wearable device and a memory device. The communication device is in data communication with the wearable device. The wearable device comprises a vibrator adjustment unit, a first bone-conducting vibrator, a second bone-conducting vibrator and a wearable device data transceiver. The vibrator adjustment unit comprises a control board, an audio power amplifier, an amplifier adjustment controller, a first vibrator socket, and a second vibrator socket. A method for interactive intervention based on biomechanical wave is also disclosed.

### CROSS-REFERENCE TO RELATED APPLICATION

This application claims priority benefit from China Patent Application No. 201810405066.7, filed on Apr. 28, 2018 in the State Intellectual Property Office of the P.R.C, the disclosure of which is incorporated herein by reference in its entirety.

### TECHNICAL FIELD OF THE INVENTION

The present application generally relates to medical systems, and more particularly, to a system and a method for interactive intervention based on biomechanical.

### BACKGROUND OF THE INVENTION

Chronic diseases such as diabetes relies heavily on passive medical drug treatment. Such treatments are generally deficient.

In addition, an interactive intervention lacks personalized treatment plan because it is difficult to constantly change an existing treatment plan with updated patient condition. Therefore, a need remains for a biomechanical interactive intervention with a data management system to prove personalized treatment for treating chronical diseases.

### SUMMARY OF THE INVENTION

The present application discloses a system and a method for interactive intervention based on biomechanical wave for providing an effective means to treat chronical disease with personalized data management service.

The system for interactive intervention based on biomechanical wave, comprising a biomechanical wave control system, a data computing device and a server.

The biomechanical wave control system comprises a communication device, a wearable device and a memory device.

The communication device is in data communication with the wearable device.

The wearable device comprises a vibrator adjustment unit, a first bone-conducting vibrator, a second bone-conducting vibrator and a wearable device data transceiver. The vibrator adjustment unit comprises a control board, an audio power amplifier coupled to the control board, an amplifier adjustment controller coupled to the audio power amplifier, a first vibrator socket coupled to the control board, and a second vibrator socket coupled to the control board. The first bone-conducting vibrator is coupled to the first vibrator socket, wherein the first bone-conducting vibrator performs biomechanical wave vibration based on a plurality of control signals, wherein the wearable device operates the first bone-conducting vibrator through the vibrator adjustment unit. The second bone-conducting vibrator is coupled to the second vibrator socket, wherein the second bone-conducting vibrator performs biomechanical wave vibration based on the plurality of control signals, wherein the plurality of control signals represents a plurality of operating amplitude and frequency of the first bone-conducting vibrator and the second bone conducting vibrator, wherein the wearable device operates the second bone-conducting vibrator through the vibrator adjustment unit. The wearable device data transceiver is in data communication with the vibrator adjustment unit and the communication device, wherein the wearable device data transceiver receives the plurality of control signals from the communication device.

The memory device stores the plurality of control signals representing a plurality of operating amplitude and frequency of the first bone-conducting vibrator and the second bone-conducting vibrator.

The data computing device is in data communication with the communication device and transmitting the plurality of control signals to the wearable device transceiver. The data computing device comprises a data input unit, a data computing device memory, a data computing device transceiver and a display. The data input unit receives a plurality of data representing disease history. The data computing device memory stores the plurality of control signals and coupled to the data input unit. The data computing device transceiver is coupled to the data computing device memory. The display is coupled to the data computing device memory.

The server remote from the biomechanical wave control system and the data computing device. The data computing device is in data communication with the biomechanical wave control system. The data computing device transceiver transmits the plurality of data to the server and receives the plurality of control signals from the server. The server transmits the plurality of control signals to the data computing device transceiver. The server comprises a data matching unit, a server central processing unit, a server memory and a server transceiver. The data matching unit matches the plurality of data with the plurality of control signals.

In various exemplary embodiments, the first bone-conducting vibrator and the second bone-conducting vibrator are configured to output the plurality of control signals through vibration of biomechanical wave at the plurality of operating amplitude and frequency.

In various exemplary embodiments, the wearable device further comprises a back strap and a waist strap, wherein the back strap is coupled to at least one of the first bone-conducting vibrator and the second bone-conducting vibrator, the waist strap is coupled to at least one of the first bone-conducting vibrator and the second bone-conducting vibrator.

In various exemplary embodiments, the wearable device further comprises a power inlet port coupled to the control board.

In various exemplary embodiments, the data computing device is a computer.

In various exemplary embodiments, the server memory stores the plurality of control signals configured to match various the plurality of data.

The method for interactive intervention based on biomechanical wave comprises: receiving, at a data computing device, a plurality of data representing disease history; transmitting, from the data computing device, the plurality of data representing disease history to a server; associating, at the server, the plurality of data representing disease history with a plurality of control signals; transmitting, from the server, the plurality of control signals to a data computing device transceiver at the data computing device; receiving the plurality of control signals at the data computing device transceiver; transmitting, from the data computing device transceiver, the plurality of control signals to a communication device of a biomechanical wave control system; transmitting, from the communication device of the biomechanical wave control system to a wearable device data transceiver, the plurality of control signals; receiving, at the wearable device data transceiver, the plurality of control signals; storing, at a memory device, the plurality of control signals; transmitting, from the wearable device data transceiver, the plurality of control signals to a vibrator adjustment unit; converting, at the vibrator adjustment unit, the plurality of control signals into amplitude and frequency data; transmitting, from the vibrator adjustment unit, the amplitude and frequency data to a first bone-conducting vibrator and a second bone-conducting vibrator; receiving, at the first bone-conducting vibrator and the second bone-conducting vibrator, the amplitude and frequency data; and operating the first bone-conducting vibrator and the second bone-conducting vibrator through vibration at operating amplitude and frequency corresponding to the amplitude and frequency data.

In various exemplary embodiments, the data computing device is a computer.

In various exemplary embodiments, the first bone-conducting vibrator and the second bone-conducting vibrator is disposed within a back strap and a waist strap.

In various exemplary embodiments, the vibrator adjustment unit is capable of adjusting the operating amplitude and frequency through an amplifier adjustment controller.

In various exemplary embodiments, the wearable device further comprises a power inlet port coupled to the first bone-conducting vibrator and the second bone-conducting vibrator.

In various exemplary embodiments, the power inlet port receives electric power supply to power the biomechanical wave control system.

Based on the above, the present application allows a biomechanical wave interactive interaction treatment to effectively treat chronic disease by adopting, personalized intervention program based on individual's historical medical condition.

### BRIEF DESCRIPTION OF THE DRAWINGS

The objects, features and advantages of the present application will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

FIG. 1 illustrates a schematic diagram of system architecture of the invention according to an embodiment of the present invention.

FIG. 2 illustrates a structural block diagram of the amplitude adjustment circuit according to an embodiment of the present invention.

FIG. 3 illustrates a schematic diagram of the key switch connection for the amplitude adjustment circuit according to an embodiment of the present invention.

FIG. 4 illustrates a schematic diagram of the main control board connection for the amplitude adjustment circuit according to an embodiment of the present invention.

FIG. 5 illustrates a schematic diagram of the connection of a class D audio power amplifier for the amplitude adjustment circuit according to an embodiment of the present invention.

FIG. 6 illustrates a schematic structural diagram of a booster circuit according to an embodiment of the present invention.

FIG. 7 illustrates a schematic structural diagram of the back strap according to an embodiment of the present invention.

FIG. 8 illustrates a schematic diagram of a waist bandage structure according to an embodiment of the present invention.

FIG. 9 illustrates a schematic structural diagram of a front of the waist strap according to an embodiment of the present invention.

FIG. 10 illustrates a schematic structural diagram of a storage bag for an intelligent controller according to an embodiment of the present invention.

FIG. 11 illustrates a schematic structural diagram of an intelligent controller cover according to an embodiment of the present invention.

FIG. 12 illustrates a schematic structural view of an intelligent controller casing according to an embodiment of the present invention.

FIG. 13 illustrates a schematic diagram of a bottom structure of an intelligent controller casing according to an embodiment of the present invention.

FIG. 14 illustrates a schematic diagram of a side wall structure of the intelligent controller casing according to an embodiment of the present invention.

FIG. 15 illustrates a second schematic diagram of the side wall structure of the intelligent controller casing according to an embodiment of the present invention.

FIG. 16 illustrates a third schematic diagram of the side wall structure of the intelligent controller casing according to an embodiment of the present invention.

FIG. 17 illustrates a schematic diagram of the back structure of the intelligent controller lampshade according to an embodiment of the present invention.

FIG. 18 illustrates a partial schematic structural diagram of the front cover of the intelligent controller according to an embodiment of the present invention.

FIG. 19 illustrates a schematic structural diagram of a front of the lampshade of an intelligent controller according to an embodiment of the present invention.

FIG. 20 illustrates a schematic diagram of a partial cooperation between lampshade and cover of the intelligent controller according to an embodiment of the present invention.

FIG. 21 illustrates a schematic structural diagram of a vibrator according to an embodiment of the present invention.

FIG. 22 illustrates a schematic diagram of a mounting position of vibrator electrode and fixing hole according to an embodiment of the present invention.

FIGS. 23-30 illustrates the changing curve of fasting blood glucose and postprandial blood glucose monitoring data before and after use of the device.

FIG. 31 is a block diagram of the system for interactive intervention based on biomechanical wave.

### DETAILED DESCRIPTION OF DISCLOSED EMBODIMENTS

Reference will now be made in detail to the present representative embodiments of the present application, examples of which are illustrated in the accompanying drawings. Wherever possible, the same reference numbers are used in the drawings and the description to refer to the same or like parts.

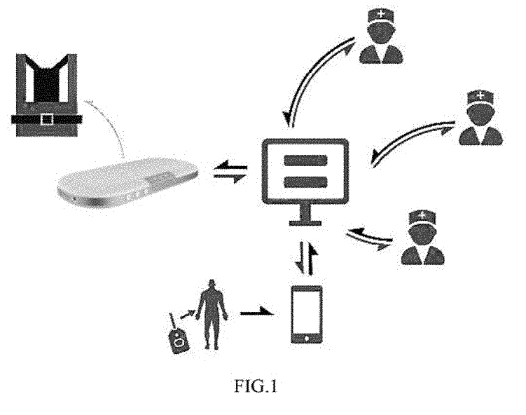

FIG. 1 illustrates a holistic view of the overall configuration of the present invention. The bio-mechanical wave interactive interaction and data management service system includes client terminal for entering user data, remote conditioning service platform, intelligent controller, and wearable device.

In operation, the client is used for entering user data. The Remote-conditioning service platform is used for receiving user data and generating and adjusting a chronic disease intervention program which includes chronic disease intervention bio-mechanical wave frequency data based on the user data, and the remote conditioning service platform dynamically optimizes the chronic disease intervention program based on the user's wearable device usage time data and corresponding efficacy. The intelligent controller is used to receive the chronic disease intervention bio-mechanical wave frequency data generated by remote conditioning service platform and outputting the vibrator control signal with the corresponding frequency. In addition, the intelligent controller is also used to record the user's wearable device usage time and upload the users wearable device usage time data to the remote conditioning service platform.

In operation, the wearable device is used for receiving the vibrator control signal generated by the intelligent controller and driving a bone-conducting vibrator to complete bio-mechanical wave vibration with corresponding frequency (frequency range 200-2000 Hz). The bio-mechanical waves are sound vibrations, which change the cell wall to rearrange the micro-fibrils of the cytoskeleton that maintain cell morphology. Initially, the plasma membrane senses the sound wave, and then the plasma membrane delivers the acoustic signal to the cell. After the receptor on the plasma membrane receives the acoustic signal, it opens the transport channel that can transport the extracellular Ca2+ into the cell. When a large amount of Ca2+ enters the cell, the cell has already converted the sound vibration signal into an ion signal that the cell can understand and transmit, these Ca2+ stimulate cells to take a series of reactions. At the same time, when the plasma membrane is transported into the cell with a large amount of Ca2+, it also opens the K+ channel. The reactive oxygen species (ROS) in the cell is also activated, the sugar concentration is also increased, and the sugar concentration in the blood is decreased. These factors together change the expression of the gene and activate the natural recovery process of the human self-organizing system. It is a natural recovery process that activates the cell structure and function of human body's self-organizing system.

The genetic material knows that it needs to carry out the next step. The final result of the cascade stimulates other proteins that can transmit information, allowing this information to transmit farther and change the transcription factors (TFs) of the cells.

Proper three-dimensional audio information waves can carry information without interference from particles or electromagnetic waves in the air. It can accurately transmit information and activate cells, improves physiological function and dysfunction of the five organs. So as long as we find the correct sound frequency, we can use it to transfer energy information to the cellular level, as the sleeping cells are awakened, the disordered cell growth begins to become orderly, the cells begin to have correctly functions, the cells regenerate, and beta cells begin to secrete insulin. The restoration of cell function has allowed the five organs to return to their natural state of life. Chronic disease is such that recover the dysfunctional function system in the exchange process of energy and information, and the overall state of the body has been fully improved.

In one embodiment, the client terminal includes a health questionnaire entry module, a physical examination data entry module, and a feedback data entry module: The health questionnaire entry module is used to fill in the health information questionnaire before the user use the product. The input information includes name, age, gender, lifestyle (daily sleep status), and health status (thirsty to drink more, big appetite, copious urine; Easily nervous, impatient, irritable; constant fatigue; headache; dizzy; memory loss, forgetfulness; reduced concentration; physical strength decrease; weight decrease; weight increase; feel ill at ease; depression; insomnia; vision decrease; lower limb pain; intermittent limp) and disease conditions (diabetes disease course; symptom descriptions and complications; insulin dosage; oral medication dosage). The physical examination data entry module is used for entering medical examination data before the user uses products. The information entered includes weight, fasting blood glucose, 2 hours after dinner blood glucose, HbA1c, triglyceride, total cholesterol, high density lipoprotein cholesterol (HDL-C), low density lipoprotein cholesterol (LDL-C), uric acid, blood pressure, urea, creatinine (Cr), insulin (fasting), Insulin (2 hours). Feedback data entry module, the user uploads feedback data to the system through the feedback data entry module according to the time required by the system (entered every day); feedback data includes: fasting blood glucose, 2 hours after dinner blood glucose, blood pressure, product used days, insulin dosage, oral drug dosage, remarks (fill in the change of their own physical signs that they found during use).

The remote conditioning service platform dynamically adjusts the decision data model and optimizes the chronic disease intervention program according to the feedback data uploaded by the user. If the user fails to upload the feedback data according to the time required by the system, the remote conditioning service platform will stop pushing the chronic disease intervention program to the user. This method can guarantee the user's feedback data and provide data basis for the continuous optimization of data models by the big data platform.

In one embodiment, the remote conditioning service platform includes a user history data visualization module for visually displaying changes in health data before and after a user receives a biomechanical wave intervention therapy. Specifically, the intelligent controller includes communication unit, main controller and vibrator driving unit. The main controller receives the chronic disease intervention biomechanical wave frequency data generated by the remote conditioning service platform through the communication unit, and outputs the corresponding enable signal. The vibrator driving unit is configured to output the vibrator control signal according to the enable signal to control the vibrator to complete the vibration of the bio-mechanical wave at the corresponding frequency.

In an alternative embodiment, the Bio-mechanical wave intelligent interactive intervention and data management service system can also provide remote expert services. Experts access the remote service platform through their clients, and platform users can interact remotely with experts through the client's APP.

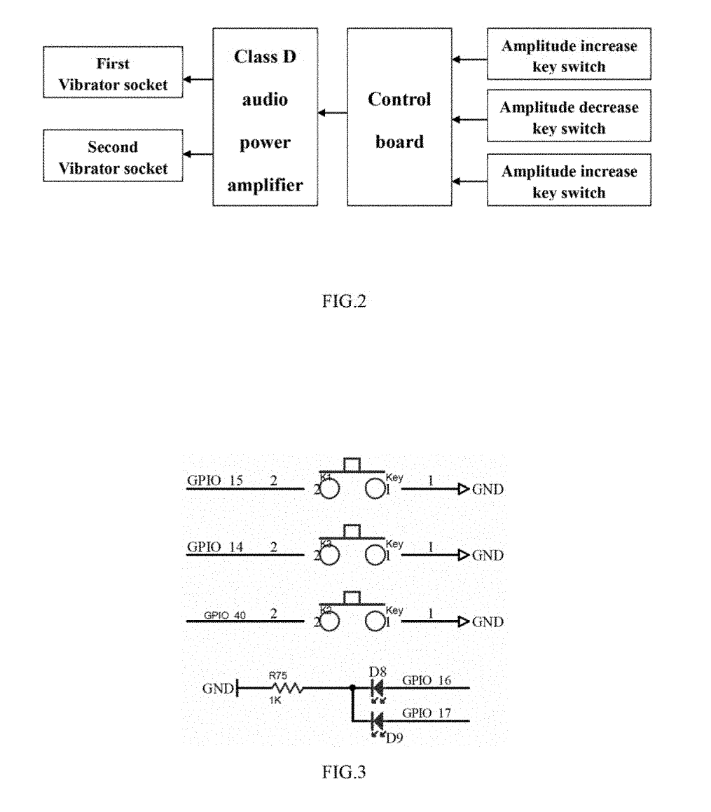

FIG. 2 and FIG. 3 illustrate a block diagram of the circuit board of the vibrator driving unit, which includes a vibrator amplitude adjustment vibrator circuit, the vibration amplitude adjustment and start/pause function can be quickly realized by pressing the button. An intelligent controller can simultaneously control the vibrator amplitude in the lumbar belt and the back belt. The user can quickly adjust the vibrator amplitude according to their own needs, which is easy to use.

The vibrator amplitude adjustment circuit includes a control board U9 (MT7688AN) and a class D audio power amplifier U3 (PAM8403), the amplitude increase button K1 (corresponding to the amplitude and button on the controller) is grounded at one end, and the other end is connected with the first sampling signal input end GPIO15 of the control board U9; the amplitude reduce button K3 (corresponding to the amplitude and button on the controller) is grounded at one end, and the other end is connected with the second sampling signal input end GPIO14 of the control board U9; Start/pause button K2 (corresponding to the amplitude and button on the controller) is grounded at one end, and the other end is connected to the third sampling signal input end GPIO40 of the control board U9;

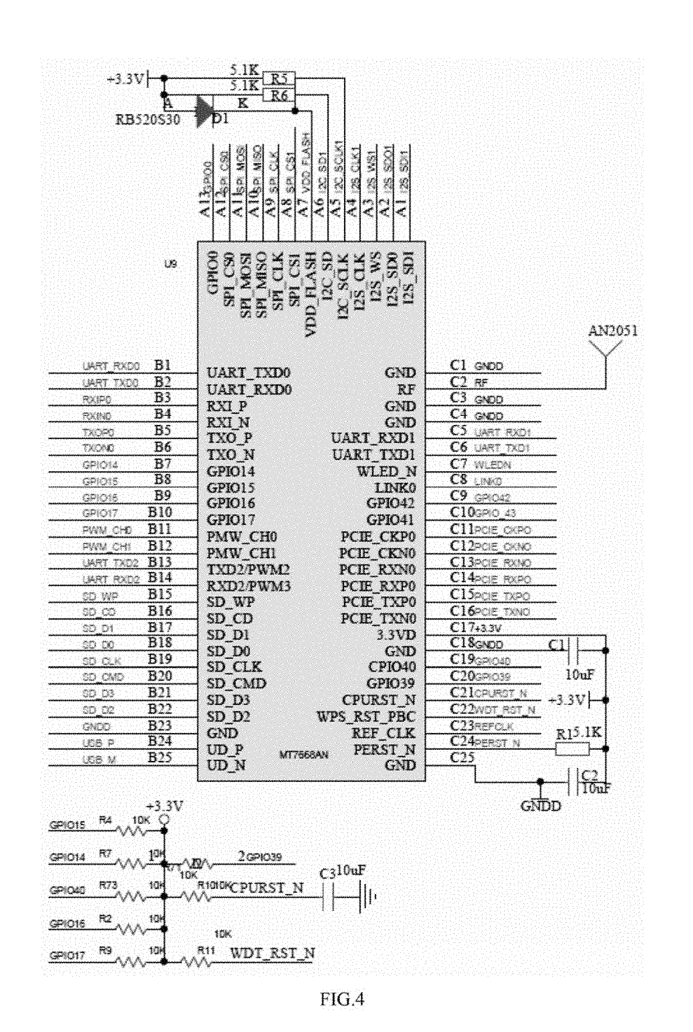

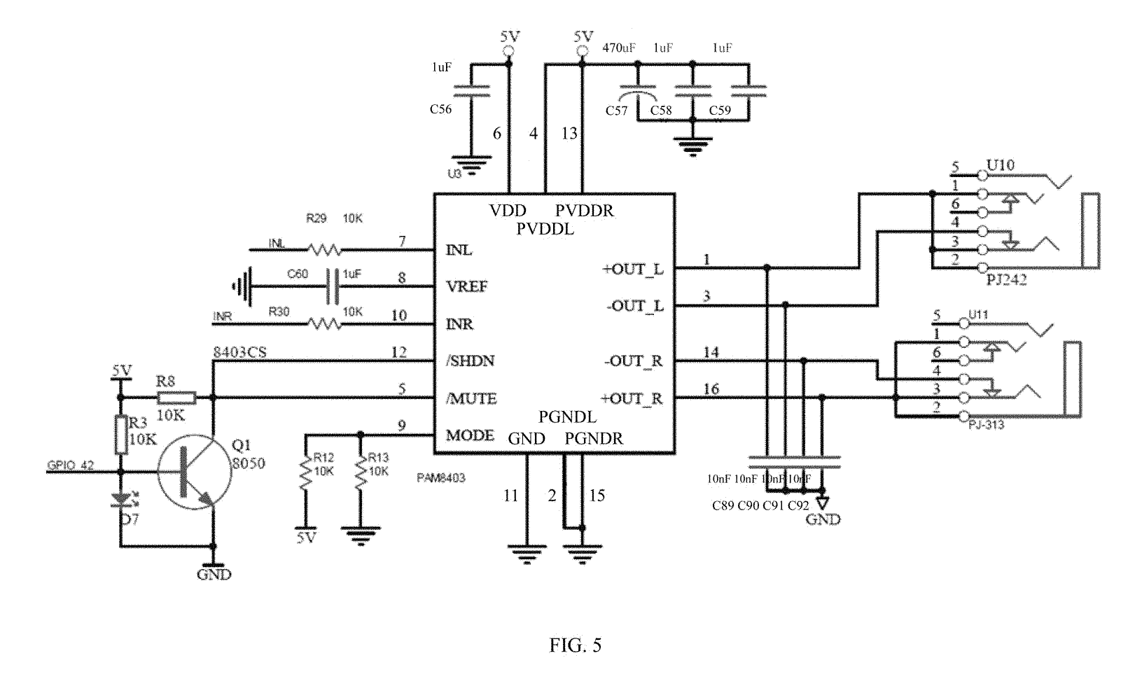

FIG. 4 and FIG. 5 illustrate the configuration of the control board. The control board enable signal output terminal GPIO42 of the control board U9 is connected to the base of the transistor Q1 (8050), the collector of transistor Q1 is respectively connected to the mute control input/MUTE pin of the Class D audio power amplifier U3 and the system shutdown control input/SHDN pin, the emitter of the transistor Q1 is grounded, and an LED D7 (LED indicator, LED is on when pin GPIO_42 is set, LED is off when set to 0) is connected between the enable signal output terminal of the control board U9 and the emitter of the transistor Q1, the base of the transistor Q1 is also connected to the collector of the transistor Q1 through a series connection of a resistor R3 and a resistor R8 (Resistance R3 and R8 have the pressure limiting current and protective effect, and only R3, R8 connected in series with Q1 and D7 can it work normally). The transistor Q1 is used for switching. When the GPIO_42 pin is set to 1, the transistor current conducts, and the two MUTE/SHDN pins are in the high state; when the pin GPIO_42 is set to 0, the transistor current is do not turn on, /MUTE, /SHDN two pins are in the low state, these two pins are low when the chip drives, otherwise does not.

The left channel output end (+OUT_L; −OUT_L) of the Class D audio power amplifier U3 is connected to the first transducer socket U10 (PJ242), and the right channel output end (+OUT_R; −OUT_R) is connected to the second transducer socket U11 (PJ313). The vibrator socket is a 3.5 mm audio socket, an intelligent controller provides two vibrator sockets, which can control the vibrator amplitude in the waist belt, and the back belt. It is easy to use.

The oscillator amplitude adjustment circuit includes a first amplitude display light emitting diode D8 and a second amplitude display light emitting diode D9. The positive electrodes of the first amplitude display LED D8 and the second amplitude display LED D9 are respectively connected to the second enable signal output terminal GPIO16 and the third enable signal output terminal GPIO17 of the control board U9. The negative poles of the first amplitude display LED D8 and the second amplitude display LED D9 are grounded through the current limiting resistor R75. Since the LED allows a small amount of current to flow, which is approximately 10 mA, a series resistor is required to limit the current.

In one embodiment, the pin of the left channel input end INL of the class-D audio power amplifier U3 is connected in series with a voltage divider resistor R29, and the pin of the right channel input end INR is connected in series with a voltage divider resistor R30. The input signal is properly divided by a resistor of about 10 k Ω to prevent distortion due to excessive signal.

In one embodiment, the positive electrode PVDDL of the left channel power supply and the positive electrode PVDDR of the right channel power supply of the class D audio power amplifier U3 are connected to the positive electrode of the power supply through a filter circuit. Specifically, the filter circuit includes an electrolytic capacitor C57, a ceramic capacitor C58, and a ceramic capacitor C59 connected in parallel to each other. The positive electrode of the electrolytic capacitor C57 is connected to the positive electrode of the power supply. The negative electrode of the electrolytic capacitor C57 is share ground with the ceramic capacitor C58 and the ceramic capacitor C59. The effect of the polar electrolytic capacitor C57 in the power supply circuit is a power filter, due to the structure of the electrolytic capacitor during use, high-frequency signals cannot be filtered out. The ceramic capacitors C58 and C59 are used to filter out high-frequency signals. The filter circuit structure has good filtering effect, can effectively filter out high-frequency clutter and ripple in the input power, improve the quality of the power supply, and improve the power supply stability of the system. In addition, the left and the right channel output terminal of the class-D audio power amplifier U3 are respectively grounded through frequency division capacitors (C89, C90, C91, and C92). The frequency division capacitor uses its high charge and low amplitude characteristics to pull the high and low pulsating current into a smooth current, the second is to place the residual AC component and harmonic pulse short circuit at the output terminal into the ground so that it cannot enter the resonator socket. The internal analog reference VREF pin is connected with a bypass capacitor C60 to GND.

In one embodiment, the intelligent controller's power supply includes a boost circuit. After boosting the input voltage, whether the intelligent controller uses battery or USB power supply, it can provide a stable 5V power supply for the vibrator to ensure stable operation of the vibrator.

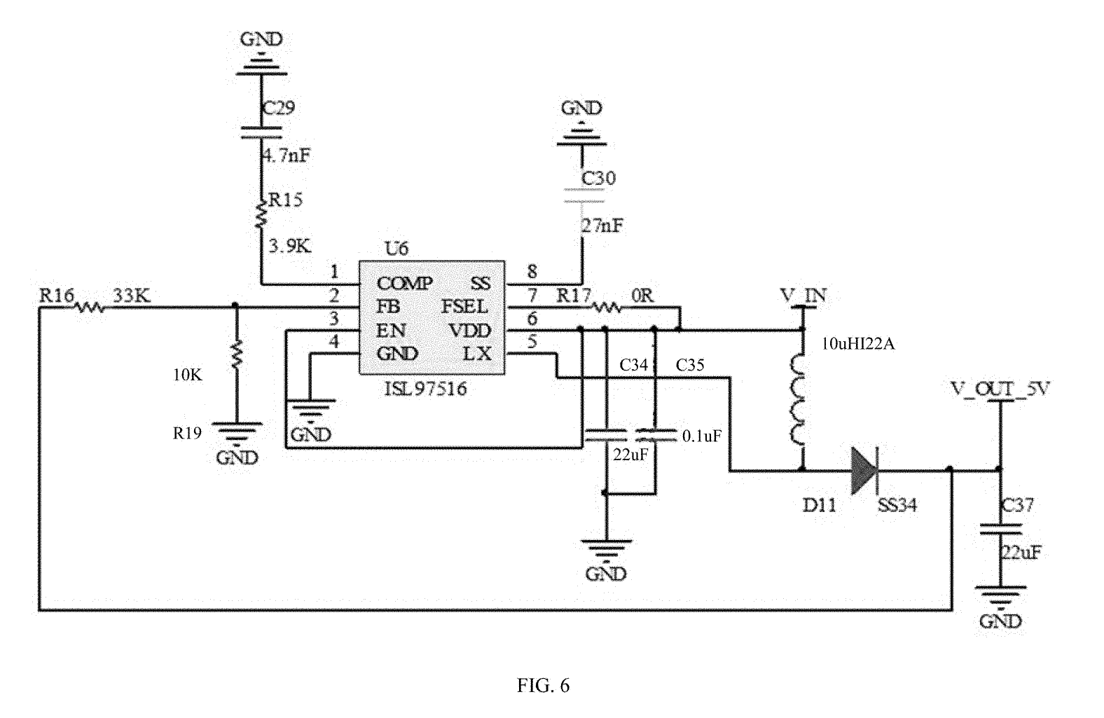

FIG. 6 illustrates a circuit configuration of the boost chip U6. The boost chip U6 uses the type ISL97516 chip, the voltage input range is 2.3-5.5V, and the conversion efficiency is as high as 90%. The first branch of the input voltage V_IN is connected to the analog, power input terminal VDD of the boost chip U6, and the second branch is connected to the frequency selection pin FSEL of the boost chip U6 through the voltage dividing resistor R17. When FSEL is low, the switching frequency is set to 620 kHz. When connected to high or VDD, the switching frequency is set to 1.25 MHz. The third branch is connected to the power switch pin LX of the boost chip U6 through the oscillating inductor L. The power switch pin LX of the boost chip U6 is also connected to the positive terminal of the boost Schottky diode D11 (SS34). The negative terminal of the boost Schottky diode D11 is connected to the boost voltage output terminal V_OUT_5V, and the oscillating inductor L between the VDD pin and the LX pin generates the oscillation harmonic, which generates a boosting action on the boosted Schottky diode D11.

The voltage boosting output is grounded through the first filter capacitor C37. The first filter capacitor C37 is used to absorb the interference signal (high-frequency interference signal) generated by the circuit board (or conductor) distributed capacitance, and improve the quality of the output voltage.

The compensation pin COMP of the boost chip U6 is grounded through the series RC spike absorption circuit. The characteristic of the capacitor is that the current through the capacitor is proportional to the voltage change rate across the capacitor, and the capacitor C29 can slowly absorb the spike voltage. The energy is consumed by the resistor R15. The RC spike absorption circuit can effectively absorb the surge caused by the switching power supply, the conduction and cut-off of the diode, and protect the circuit.

The voltage feedback pin FB of the boost chip U6 is connected to the boost voltage output through a feedback resistor R16. The off control pin EN of the boost chip U6 is connected to the analog power input VDD. The voltage feedback pin FB of the boost chip U6 is also grounded through the balancing resistor R19.

The soft start control pin SS of the boosting chip is connected in series to the soft start capacitor C30. The soft start capacitor C30 can bypass the AC interference and can integrate the voltage so that the SS terminal voltage rises slowly.

In one embodiment, the input voltage V_IN is connected to the analog power input VDD of the boosting chip through a filter circuit. Specifically, the filter circuit includes a second filter capacitor C34 and a third filter capacitor C35 connected in parallel with each other, and the second filter capacitor C34 and the third filter capacitor C35 are connected in parallel and share the ground. The large-capacitance capacitor C34 connected in parallel is used to filter out low-frequency signals and the small-capacity capacitor C35 is used to filter out high-frequency signals. The filtering effect is good, high and low frequency of the clutter and ripple in the input voltage can be effectively filtered out.

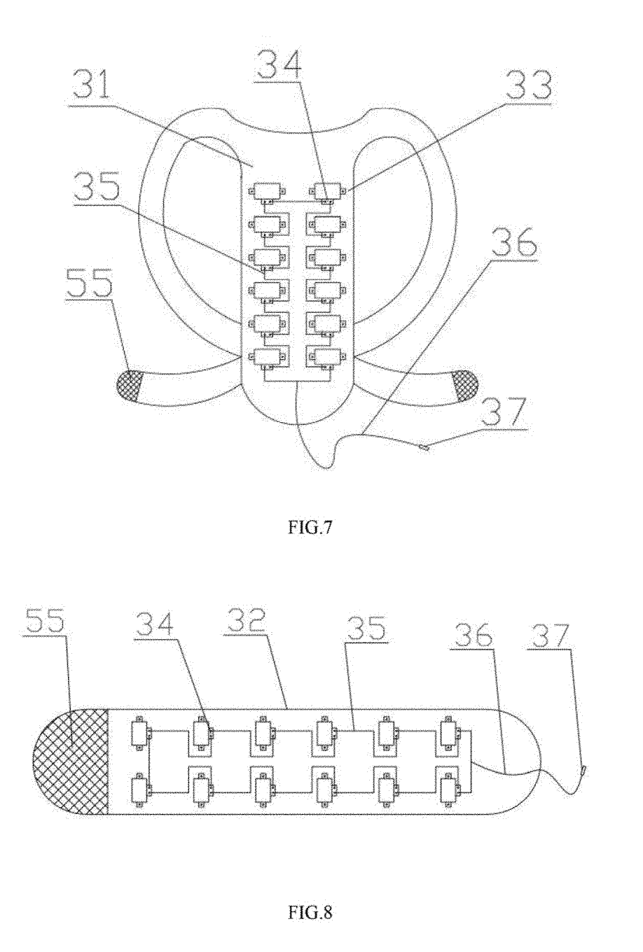

FIG. 7 and FIG. 8 illustrate structural views of the wearing device. The wear device includes a back strap (31) and a waist strap (32). For convenience of wearing, the Velcro (55) is provided on the back strap (31) and the waist strap (32). The back strap (31) and the waist strap (32) are all provided with a vibrator string of bone conduction oscillator. The bone conduction vibration element on the back strap (31) is distributed on both sides of the human spine and around the body's pulse. Each vibrator string includes at least 12 vibrators (33). The vibrator (33) is provided with an electrode (34) for wiring, the electrode (34) is connected in series with the wire (35), and the wire (35) is connected to the power plug (37) through a outgoing line (36); The intelligent controller is provided with two vibrator sockets (13) matched with the power plug (37). The length of the outgoing line (36) is relative to the distance from the beginning of the outgoing line (36) to the vibrator socket (13). The length of the vibrator outgoing line (36) on the back strap (31) and the waist strap (32) is set according to the position of the intelligent controller storage bag (38). When users wear the wearable device, the length of the two outgoing line (36) is just enough to insert the power plug (37) into the vibrator socket (13) of the intelligent controller that in the storage bag (38). The outgoing line (36) is highly conformable to the human, body, and without the problem of line winding.

FIG. 9 illustrates a appearance design of a waist belt and FIG. 10 illustrates a structural view of an intelligent controller storage bag. The intelligent controller storage bag (38) is provided on the front face of the waist belt (32). The intelligent controller storage bag (38) is disposed on the waist belt (32) through a suture. An elastic belt (39) is arranged at the opening of the intelligent controller storage bag (38). One side of the intelligent controller storage bag is provided with a charging interface hole (40) and a power switch control hole (41) cooperating with the intelligent controller. The other side is also provided with a start/pause control hole (42) and an amplitude adjustment hole (43) cooperating with the intelligent controller. As the result, the controller does not need to be taken out for performing the normal operation.

FIG. 11 illustrate a side view of the cover plate, the intelligent controller includes a cover plate (1) and a casing (2). The periphery of the cover plate (1) is provided with a concave inner sealing wall (3), and the inner sealing wall (3) is provided with a plurality of clamp parts (4).

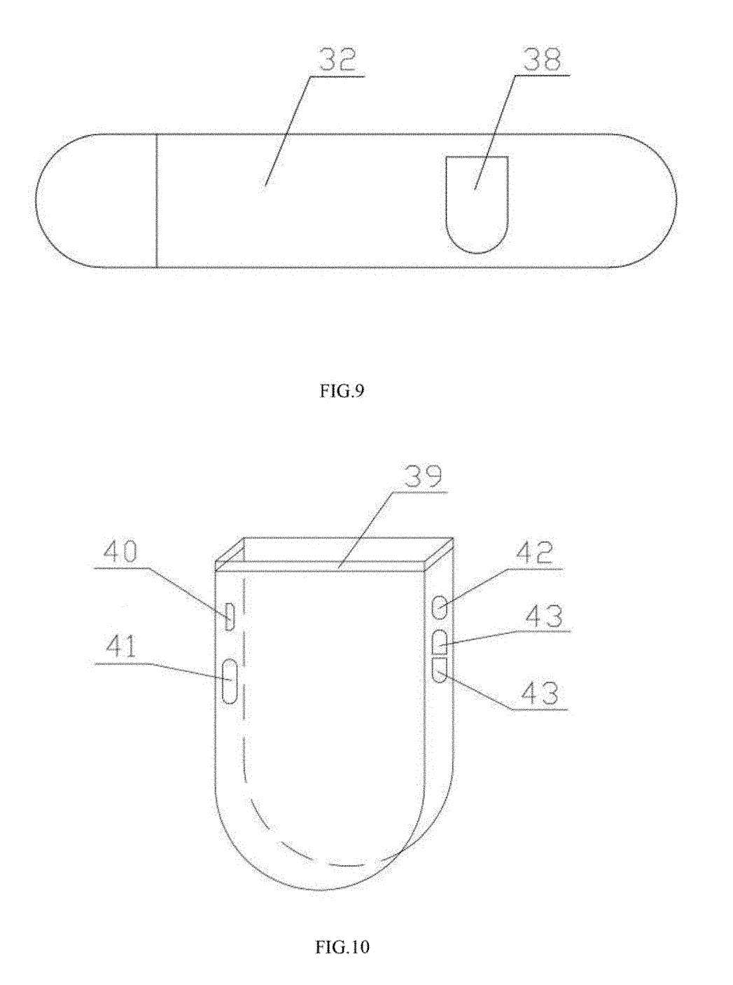

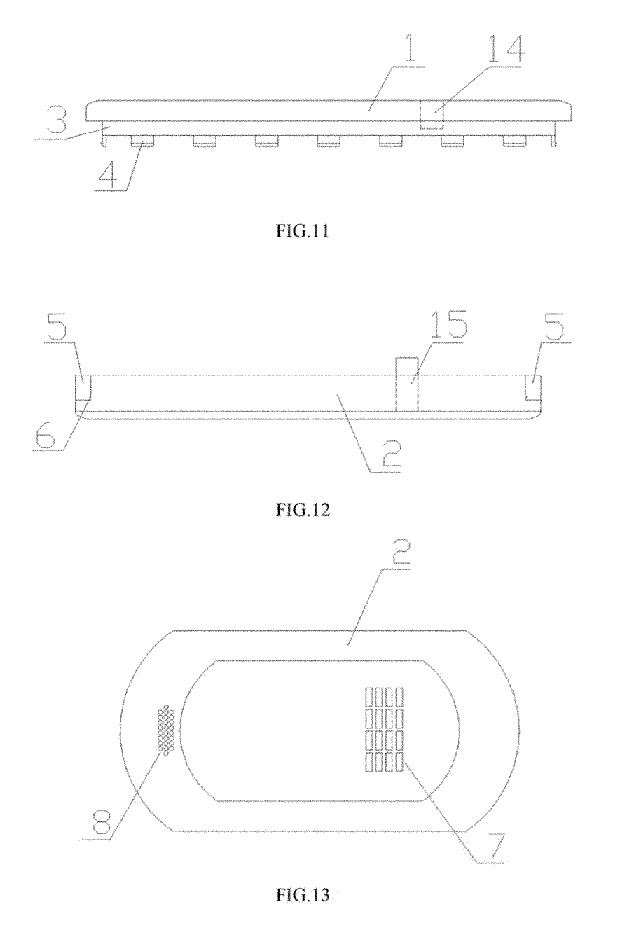

FIG. 12 illustrates a side view of a controller casing. A periphery of the casing (2) is provided with a step to form an outer sealing wall (5) that cooperates with the inner sealing wall (3), and the outer sealing wall (5) is provided with a s snap-fitting portion (6) that cooperating with the clamp parts (4).

FIG. 13 illustrates a front view of the bottom surface of a controller casing. A heat dissipating through-hole (7) is provided on the bottom surface of the casing (2) in cooperation with the mainboard power supply, an acoustic hole (8) is provided in the position matching the mainboard audio output unit.

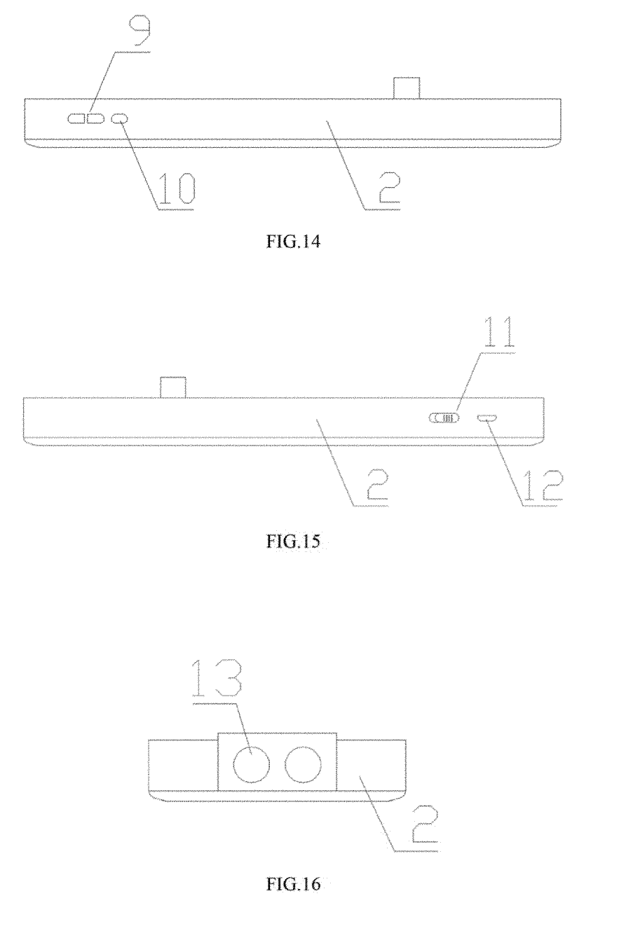

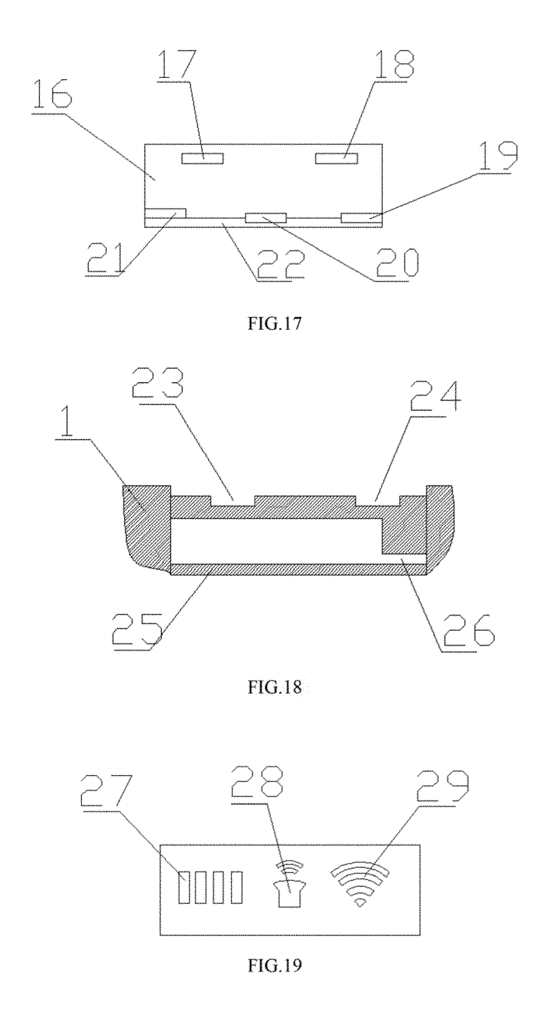

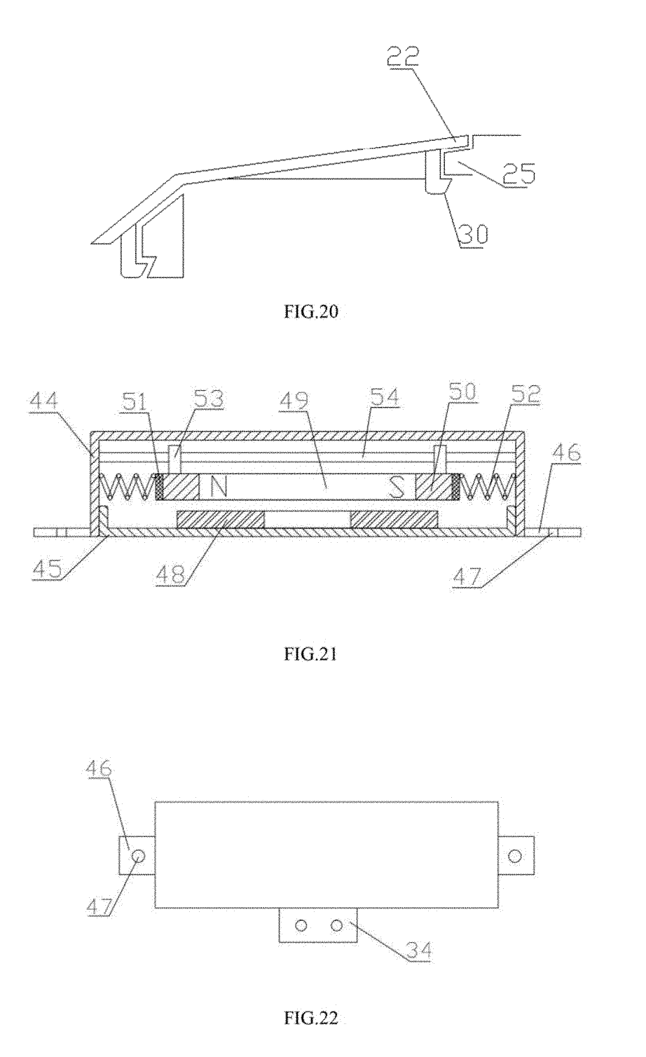

FIG. 14˜16 illustrate three side views of the sidewall of a controller casing. The side wall of the casing (2) is provided with an amplitude adjustment button (9) a start/pause button (10), a power switch button (11), a charging interface (12) and two vibrator outlets (13). The number of the vibrator outlets (13) is two, and one intelligent controller simultaneously connects two sets of vibrators, and supplies power for two sets of vibrators at the same time, one set for the back of the human body and one set for the waist.

The cover plate (1) is also provided with at least one pre-positioning cylinder (14), and the casing (2) is provided with a pre-positioning column (15) cooperating with the pre-positioning cylinder (14), the end of the pre-positioned column (15) protrudes the casing (2) (Shown in FIG. 11 & FIG. 12). The pre-positioning post that protrudes the casing is matched with the pre-positioning cylinder on the cover plate, and the pre-positioning before the clamp can be achieved, so that the clamp parts and the snap-fitting portion can be more accurately aligned and completed to be installed.

In one embodiment, the prepositioning cylinder (14) is disposed at a non-center symmetrical point of the cover plate (1), and the pre-positioning column (15) is disposed at a non-center symmetrical point of the casing (2). For the symmetrically-structured upper casing, the pre-positioning, column/cylinder installation position is a non-center symmetrical point, which can achieve the purpose of preventing reverse connection and is more convenient to use.

FIG. 17˜20 illustrate the packaging, structure of a lampshade. The lampshade (16) is provided on the cover plate (1). The back of the lampshade (16) is provided with buckles (17, 18, 19, 20) and a positioning block (21) integrated therewith. The bottom of the lampshade (16) is provided with an extended sealing edge (22), the front surface of the cover plate (1) is provided with card slot (23, 24), a snap-fitting table (25) and a positioning groove (26) cooperating with the positioning block (21), the width of the snap-fitting table (25) cooperates with the width of the sealing edge (22), the buckles (17, 18) are engaged with the card slots (23, 24), and the buckles (19, 20) are engaged with the snap-fitting table (25). A transparent electricity indication area (27), a transparent oscillator amplitude indication area (28) and a transparent wireless signal indication area (29) are arranged on the front of the lampshade (16).

In operation, the installation process of the lampshade follows the following steps: First, the buckles (17, 18) are buckled with the card slots (23, 24). Then, pressing the one end of the buckle (19, 20). The snap-fitting table (25) is in contact with the outer corners of the buckles (19, 20) first. Because the outer corners (30) have rounded corners, the snap connection process is gentler and the buckles are not easily damaged. After the buckle (19, 20) is engaged with the snap-fitting table (25), the positioning block (21) falls into the matching positioning groove (26). The positioning block (21) and the positioning groove (26) can achieve the limitation in multiple directions of the lampshade (16), so that the fixing effect of the lampshade (16) is more obvious. At the same time, the width of the sealing edge (22) just overlaps with the snap-fitting table (25) to achieve a good seal. After the package is completed, the front surface of the lampshade (16) and the front surface of the cover plate (1) will remain completely flat and easy to use.

In one embodiment, the external comers (30) of the buckles (17, 18, 19, 20) are rounded, so that it is easier to install and remove the lampshade, and the buckles are not easily broken at the same time.

FIG. 21 and FIG. 22 illustrate structural view of the vibrator and illustration of its installation. The vibrator (33) includes an upper casing (44) and a lower casing (45). A fixing piece (46) extends from both sides of the upper casing (44). The fixing piece (46) is provided with a fixing hole (47). The sewing thread stitches the vibrator (33) to the wearable device through the fixing hole (47) in the fixing piece (46); the upper casing (44) and the lower casing (45) cooperate with each other to form a closed space. The closed space is provided with a primary and a secondary. The primary includes a coil (48) fixed inside the lower casing (45), and the coil (48) extends out of the electrode (34) on the outside of the lower casing (45). The secondary includes a permanent magnet (49) disposed directly above and parallel to the coil (48), a mass block (50) fixedly attached to both ends of the permanent magnet (49), a buffer plate (51) fixed on both sides of the mass block (50), the springs (52) connected to the two inner walls of the upper casing (44) are respectively fixed by mass block (50), a limit ring (53) fixed above the two mass blocks (50), and a limit rod (54) matched with the limit ring (53) and fixedly connected between two inner side walls of the upper casing (44). Add the limiting device so that the permanent magnet (49) can only vibrate transversely, which not only avoids the problem of noise caused by hitting the upper and lower casing, but also reduces the problem of damage to the vibrator due to collision.

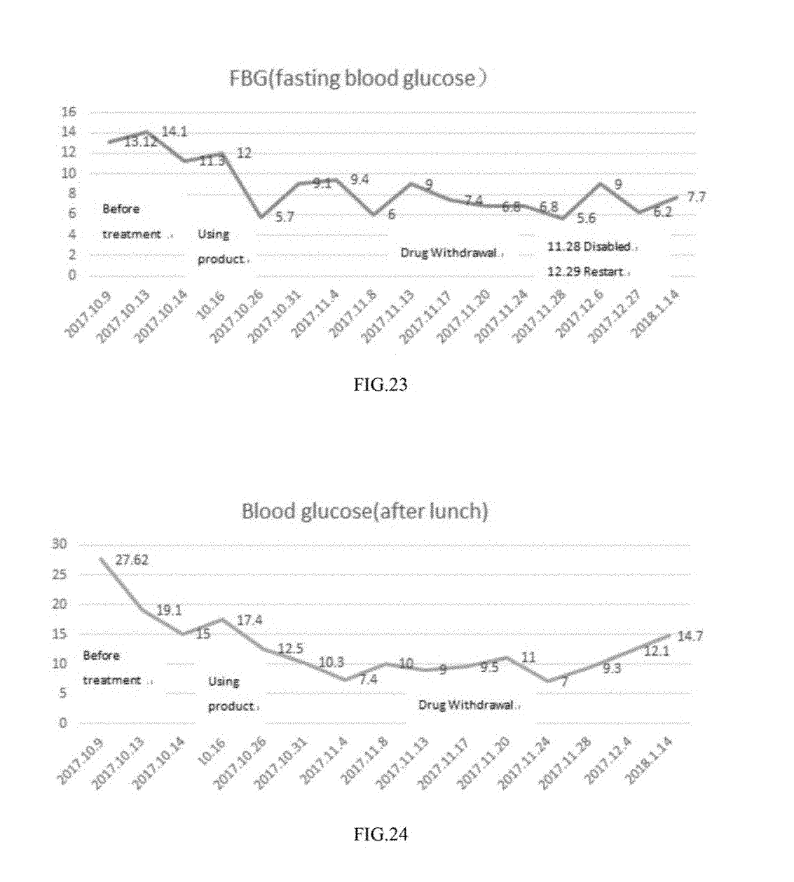

FIG. 23˜30 illustrate the change of fasting blood glucose level and postprandial blood glucose monitoring data before and after using the device in 2-dimensional diagrams.

FIGS. 23 and 24 illustrate Patient 1's change of fasting blood glucose level and postprandial blood glucose monitoring data before and after using the device. Patient 1's fasting blood glucose was 13.12 mmol/l, and postprandial blood glucose was 27.62 mmol/l on Oct. 9, 2017. Patient 1 started use the wearable devices on Oct. 16, 2017, the blood glucose level decreased gradually. The use of the device began to stop the medicine after half a month. During the period from November 28 to December 28, when the device was deactivated and the medicine was not taken, blood glucose also remained stable. And verify the stability of the device's efficacy again. On December 29, Patient 1 restarted use the device. In the absence of any medication, fasting blood glucose was 7.7 mmol/l after half a month of restarted use the device. The user has been completely rid of the medicine after using the device, and the blood glucose gradually returns to normal.

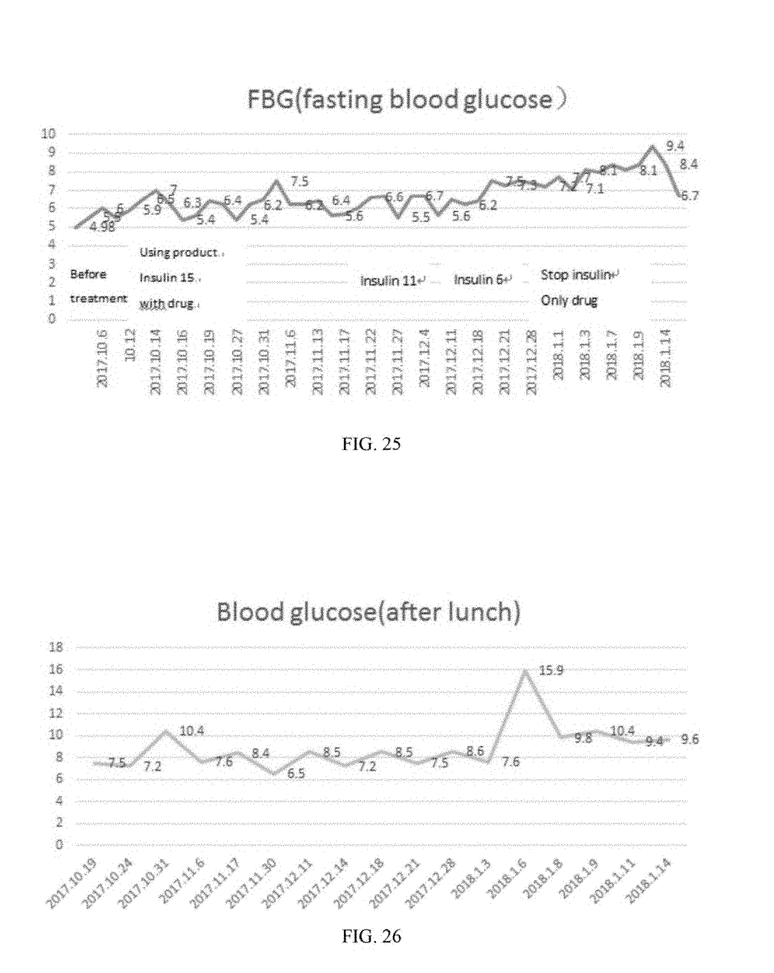

FIGS. 25 and 26 24 illustrate Patient 2's change of fasting blood glucose level and postprandial blood glucose monitoring data before and after using the device. Patient 2's fasting blood glucose was 4.98 mmol/l and insulin was 15 units with oral medicine on Sep. 26, 2017. Patient 2 started use the device on Oct. 12, 2017. During this period, blood glucose remained stable and insulin was reduced to 11 units after one month of use the device. After two months, insulin was discontinued, only oral medicines, fasting blood glucose and postprandial blood glucose were remained stable. So far, it has been more than six months and glycosylated hemoglobin is 7.6 mg/dl.

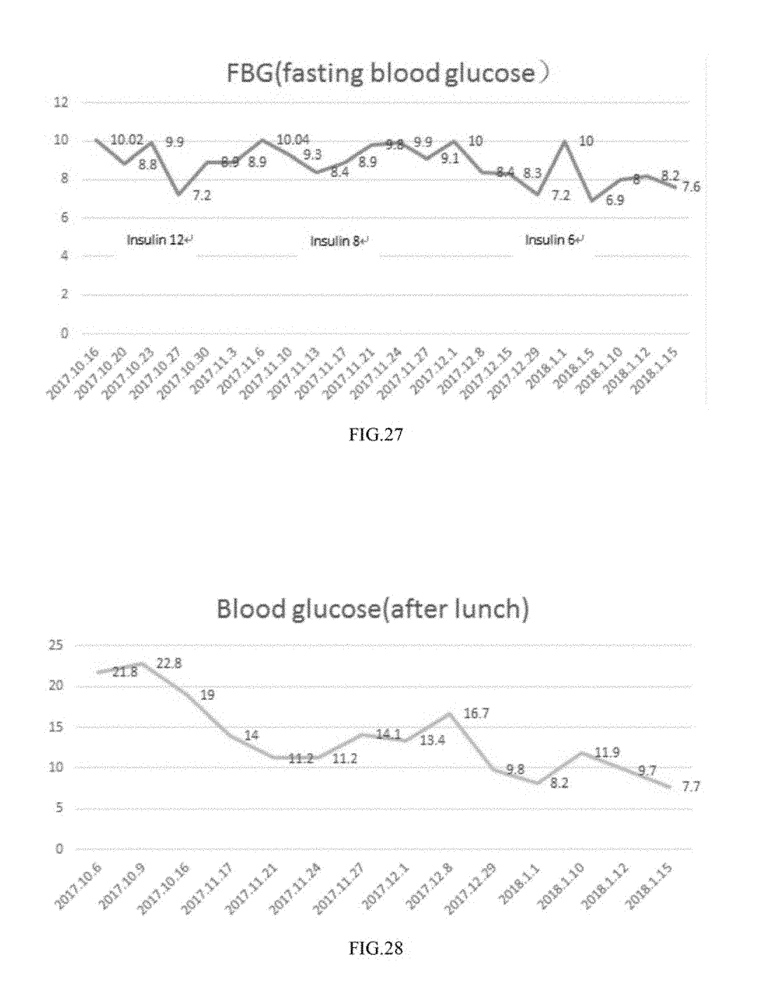

FIGS. 27 and 28 illustrate Patient 3's change of fasting blood glucose level and postprandial blood glucose monitoring data before and after using the device. Patient 3's fasting blood glucose was 12.48 mmol/l and postprandial blood glucose was 25.9 mmol/l before use the device. Patient 3 started use the device on Oct. 16, 2017. During the period of using, the amount of insulin was gradually reduced, and the fasting and after lunch blood glucose were shown to decrease. After three months, insulin was reduced to 3 units, fasting blood glucose was maintained at about 7 mmol/l and postprandial blood glucose was at about 10 mmol/l.

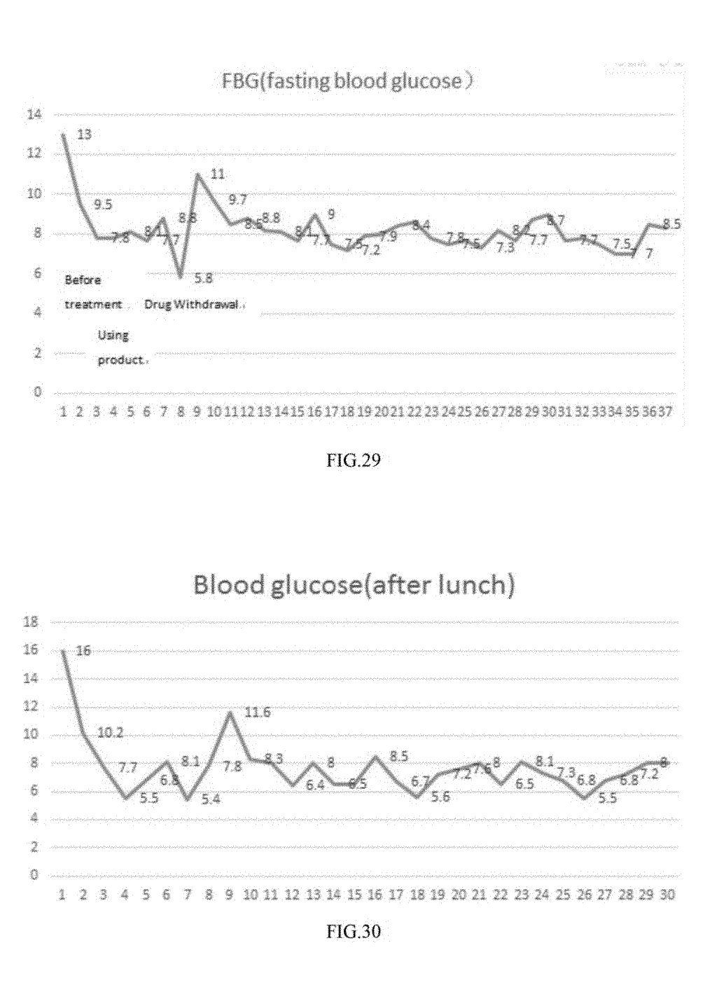

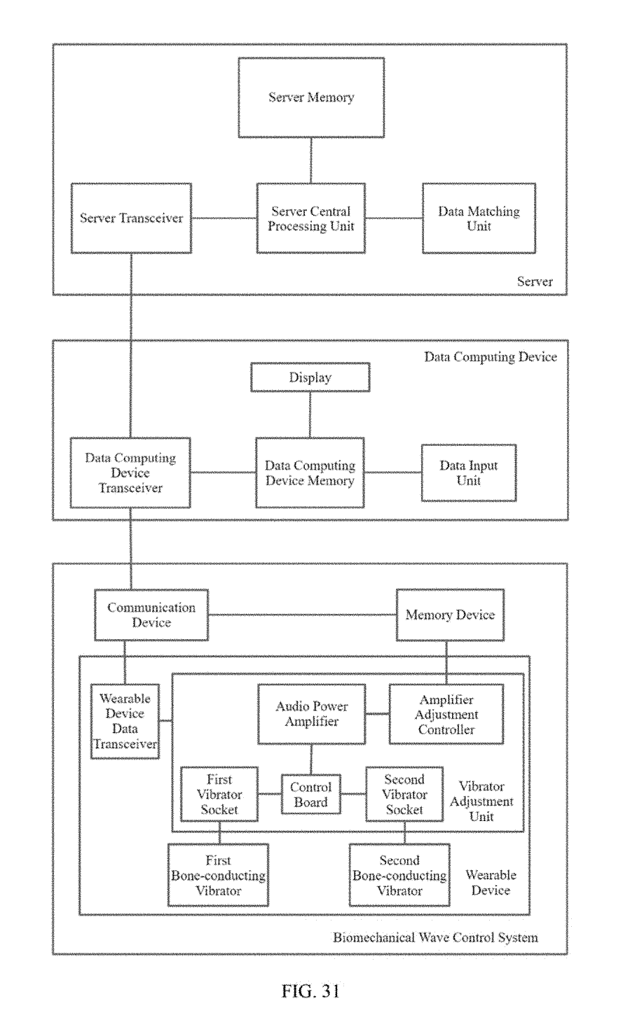

FIGS. 29 and 30 illustrate Patient 4's change of fasting blood glucose level and postprandial blood glucose monitoring data before and after using the device. Patient 4's fasting blood glucose was 13 mmol/l. After starting the device about one week, the blood glucose began to decrease, stopping use medicines, fasting and postprandial blood glucose remained stable. As the result, the user has completely got rid of the drug.

Additional evidence are also provided. Patient 5 used the device about 3 months. Before Patient 5 start use this device, Patient 5 had use insulin for 8 years, daily injection of 15 units, Patient 5's fasting blood glucose was 7 mmol/l. After using the device, insulin dosage is reduced by 10 units, fasting blood glucose falls below 6 insulin has been stopped for 4 years. Patient 6 has used the device for 1 year, taken medicine for 6 years before use this device. After wearing the device for 2 months, the fasting blood glucose decreased from 10.4 mmol/l to 6.4 mmol/l. Patient 7 used for 2 months, injected insulin for 5 years before use it; after using, gradually reduce the amount of insulin, the hypertension drug was reduced by ¼, blood pressure and blood glucose levels remained at the level of medication, and insulin was disabled. Patient 8 used for 1 month, has taken medicines of dimethyldiguanide and NovoNorm for 5 years; Patient 8 stopped taking the medicines after using the device for 15 days; and after 18 days, glucostasis is basically identical to the drug application level.

The following nine application examples provide further evidence: Among patients, five use the device for 35-40 days, two for 2 months, 1 for nearly 3 months, while 1 for over 2 years. Patients are selected in a completely random manner. It is noted that the average of measured values for each stage is taken; and the size of effective sample data should be larger than 25 measurements each month.

Patient 9, 76 Years Old; 6 Years of Medical History; Drug Withdrawal; Efficient. After long-term medication of dimethyldiguanide and NovoNorm, Patient 9 began to use the wearable acoustic wave suit on June 2 and kept wearing it for 693 hours. After 15 days, Patient 9 stopped taking the dimethyldiguanide; otherwise, NovoNorm was taken twice every day. After 18 days of drug withdrawal, glucostasis is basically identical to the drug application level. According to the relevant data, the maximum value is 8.3 which appears twice; while the minimum value 6.8 occurs once. Other values are around 7. The average level of three days before adoption of the acoustic wave: 6.9 (fasting blood-glucose); The average level of three days before adoption of the acoustic wave: 14.3 (after breakfast); The average level after drug withdrawal: 6.9 (fasting blood-glucose); Before drug withdrawal (medications plus acoustic wave): 6.59 (fasting blood-glucose); The average blood glucose level of 28 days after dimethyldiguanide withdrawal and acoustic wave adopted: 12.83 (after breakfast).

Patient 10 58 Years Old; Taking Medicines for 3 Years; Stopping Using the Drugs. On May 22, 2016, Patient 10 stopped to use drugs and began to employ the acoustic wave on May 26 for 959 hours. During 37 days of drug withdrawal, blood glucose in the process of acoustic wave adoption is stable and identical to that during taking drugs. The highest fasting blood-glucose level is 8.2 and only takes place once; and it is around 7 in most cases. As the observation period only lasts for 2 months, decline process for the blood glucose to drop to 6 and below cannot be observed. In addition, insufficient wearing time has an impact on its effects.

According to detections for three days before the adoption of acoustic wave, fasting blood-glucose level is 7.96, while 8.73 and 10.5 for the blood glucose level after breakfast and lunch respectively. Fasting blood-glucose level during the employment of acoustic wave (drug withdrawal) is 7.38; while respectively 10.3 and 10.7 for the blood glucose level after breakfast and lunch as well.

Patient 11, 62 Years Old; 8 Years of Medical History; Never Taking Drugs; Efficient. On May 27, 2016, Patient 11 began to wear the acoustic wave suit for 913 hours. As a result, the blood glucose level declined and his weight dropped. Due to hot weather and no air-conditioner, wearing time is insufficient. If the observation can continue for 3 months, the blood glucose level can be brought down to about 6. According to detections for three days before the adoption of acoustic wave, fasting blood-glucose level on average is 8.53 (9.1 for the highest level), while 14.16, 11 and 13.6 for the blood glucose level after breakfast, lunch and dinner respectively. After acoustic wave adopted, the fasting blood-glucose level is 7.75 (an average value of 28 days) with a maximum value of 8.2. After breakfast, lunch and supper, the blood glucose levels are 12.14, 10.47 and 10.91 separately.

Patient 12, 53 Years Old: 5 Years of Medical History; Drug Withdrawal; Efficient. Patient 12 began to use this acoustic wave system on May 27, 2016 and stopped to take dimethyldiguanide and gliclazide on June 15 and 28 respectively. The acoustic wave system was used by him for 1,541 hours in total. After withdrawal of all drugs, Patient 12's blood glucose level changes steadily and is in basic consistency with that during drug taking. After drug withdrawal, the highest blood glucose level 8.3 appears for three times, while it lingers around 7 for other periods. Patient 12 is able to keep using this system for 3 months continuously, it is likely for the blood glucose level to further go down. The blood glucose levels of three days before acoustic wave is used are 7.4 for FBG, 8.33 after breakfast, 11.76 after lunch and 9.9 after supper. In comparison, on the 28th day after acoustic wave is used (containing the average blood glucose level during drug withdrawal), it is 7.8 for FBG (an average value of 28 days), 11.35 after breakfast, 9.45 after lunch and 10.12 after supper.

Patient 13, 63 Years Old; 8 Years of Medical History; Taking Dimethyldiguanide Three Times A Day in the Long Term; Drug Withdrawal; Good Effect. Since May 27, 2016, the time of wearing acoustic wave is 1,222 hours, Patient 13 does not take exercises and diets remain the same. On June 13 when Patient 13 ceased to take all drugs, the blood glucose level still remained below 7 accompanied by obviously improved constipation and weight reduction. The average blood glucose level of three days before acoustic wave treatment is 6.6 for FBG, 9.26 after breakfast, 9.26 after lunch and 9.2 after supper. After the acoustic wave is adopted, the average blood glucose levels of FBG, after breakfast, after lunch and after supper, are 6.32 (an average value for 28 days), 10.11, 9.2 and 9.5 correspondingly.

Patient 14, 72 Years Old; Hypertension & Hyperglycemia; Taking Hypotensive Drugs for 15 years; Significant Effects. At the time of accepting the acoustic wave treatment system, Patient 14's blood pressure was up to 200 and above. During 15 years of taking hypertensive drugs, Patient 14's physical condition was extremely poor and Patient 14 felt morbus asthenicus and dizzy. On May 25, 2016, Patient 14 began to wear the acoustic wave suit for 1,697 hours. After two weeks, the blood glucose and the blood pressure dropped to about 5.5 and 140 respectively. Within, one and a half months, the hypertensive drugs that Patient 14 took were a quarter of the original dosage, accompanied by tinnitus disappearance, weight reduction and significantly improved mental status. Before the adoption of acoustic wave, FBG is 7, the blood glucose after breakfast is 11, while that after lunch or supper is 4.5 or 12.08 separately. At the 28th day of acoustic wave adoption, FBG becomes 5.28 and the blood glucose after breakfast or supper is 9.54 or 10 and below correspondingly.

Patient 15, 76 Years Old; 20 Years of Medical History; Taking insulin for 10 Years with Zero C-Peptide, and Injected 33 units Each Day; 10 Units of Dosage Reduction Generating Favorable Effects. Patient 15 began to use acoustic wave since May 25, 2016, lasting 1,697 hours. Within gradual increase in insulin dosage, leg ulcers appear. Patient 15 tried multiple methods which turned out to be in vain. Each time Patient 15's therapy is changed, his condition worsened and had to be hospitalized. Within one month, 10 units of insulin dosage are decreased and the blood glucose slowly drops to 7 and below after two months. Patient 15 is firmly confident that he should keep taking this acoustic wave system for three months. In this process, due to reasons such as forgetting injections and having sweetmeat, etc., Patient 15's blood glucose varies, but can rapidly go down. This is an extremely significant breakthrough. In addition, Patient 15's body begins to secrete autologous insulin. Before the adoption of acoustic wave (insulin is injected three times a day), the FBG is 13 while the blood glucose levels after breakfast, lunch and supper are 12.8, 13.8 and 13.03. After the acoustic wave is used, FBG is 7.37 (before dosage reduction of the first time) and 6 units of insulin are injected every day. Moreover, blood glucose levels after breakfast, lunch and supper are 9.7, 10.91 and 10.22, respectively. After dosage reduction for the first time, FBG is 8.12, while blood glucose levels after breakfast, lunch and supper are 13.46, 10.68 and 11.8.

Patient 16, 60 Year Old; 6 Years of Medical History; Insulin Injected for 4 and a Half Years; Taking Hypotensive Drugs for 20 Years. Patient 16 began to wear acoustic wave suit since Apr. 19, 2016 and two units of insulin are reduced each time. Up to June 13, insulin injection is completely ceased and Patient 16 only took three quarters of the original hypertensive drug dosage. In addition, Patient 16's blood pressure and glucose are both within a normal range. At present, insulin injection has stopped for nearly two months. Patient 16's blood glucose is basically stable. However, in July, Patient 16 went out to travel; despite of hyperphagia, Patient 16's blood glucose is still fundamentally steady and the blood pressure remains at around 140.

Patient 17, 76 Years Old; 8 Years of Medical History; Taking dimethyldiguanide for 6 years; Hypertension, Cataract, Edema, Weakness, and Aconuresis. Patient 17 began to receive drug withdrawal before the adoption of acoustic wave in April 2014. Up to now, acoustic wave has been applied on Patient 17 for over 10,000 hours. Since 2008, Patient 17 started to take dimethyldiguanide and Extended Release Nifedipine Tablets. In 2013, Patient 17's pathogenetic condition became worsen expressed in general dropsy, rises of blood pressure and glucose, weakness and frequent urinary incontinence. Besides, it was extremely difficult for Patient 17 to walk on her own. Concerning Patient 17's blood pressure and glucose, the former was usually 180 and above, while the latter had a level of 9.6 and above on the premise of taking drugs. At the first stage of acoustic wave treatment, Patient 17's blood glucose dropped from 10.4 on April 16 to 7.4 on June 9 on one hand; on the other hand, the blood pressure went down to 166/80 from 180. In addition to the disappearance of leg lumps, edema is relieved, physical power obviously enhanced and aconuresis cured. In 2015, Patient 17 insisted on using the acoustic wave system all year round so that her blood pressure was recovered to a level of 142 and the average blood glucose level became 6.5 accompanied by complete disappearance of aconuresis. Thanks to edema vanishing and weight reduction of 10 kg, Patient 17 was able to walk for 3,000 m without feeling any fatigue. The current blood pressure and blood glucose indexes have both returned to the normal level of youngsters. Together with the heal of complications, Patient 17 felt like 20 years younger. Based on such a continuous acoustic wave treatment lasting more than 10,000 hours, it is proven to be a diabetes rehabilitation method without any side effect and beneficial to health.

Based on our clinical observations over the years, hypoglycemic effect is almost 100% effective provided that patients persevere in wearing the acoustic wave devices.

First, for patients with blood glucose not high and drugs not taking, their blood glucoses are able to be stabilized at 7 and below around two weeks. The duration of three months is a period of treatment.

Second, for patients taking oral drugs or insulin injections, it is safer to reduce dosage gradually so that fluctuations of blood glucose are not too significant. Around one to two month(s), patients can completely stop to take drugs. After drug withdrawal, patients are required to keep wearing the acoustic wave suit for half a year (a period of treatment).

Monitoring data and clinical results all demonstrate that acoustic wave has an obvious regulating effect on insulin and blood glucose levels. Up to now, our clinical applications preliminarily prove that it is positive that the dynamic repair treatment system is able to take the place of drugs or insulin to play a role in reducing blood glucose. Meanwhile, the relevant complications are relieved or healed; most patients have their weights dropped and nearly all of them have a feeling that their overall physical conditions have been improved. In a word, this system is secure and efficient. Not only should it be another choice of diabetes treatment approaches in the future, but it is good news for patients with diabetes and metabolic syndrome.

The above description is only a preferred embodiment of the invention. It should be understood that the invention is not limited to the forms disclosed in here, and should not be taken as other excluding embodiments, but can be used in various other combinations, modifications, and environments. It can be modified within the scope of the concepts described in this document by the teachings above or in the related field. Modifications and changes made by those people in the field without departing from the spirit and scope of the invention shall belong to the protection scope of the appended claims.

It will be apparent to those skilled in the art that various modifications and variations can be made to the structure of the present application without departing from the scope or spirit of the present application. In view of the foregoing, it is intended that the present application cover modifications and variations of this application provided they fall within the scope of the following claims and their equivalents.

What is claimed is:

1. A system for interactive intervention based on biomechanical wave, comprising:
a biomechanical wave control system comprising:
a communication device;
a wearable device, wherein the communication device is in data communication with the wearable device, the wearable device comprises:
a vibrator adjustment unit comprising:
a control board;
an audio power amplifier coupled to the control board;
an amplifier adjustment controller coupled to the audio power amplifier;
a first vibrator socket coupled to the control board; and
a second vibrator socket coupled to the control board;
a first bone-conducting vibrator coupled to the first vibrator socket, wherein the first bone-conducting vibrator performs biomechanical wave vibration based on a plurality of control signals, wherein the wearable device operates the first bone-conducting vibrator through the vibrator adjustment unit;
a second bone-conducting vibrator coupled to the second vibrator socket, wherein the second bone-conducting vibrator performs biomechanical wave vibration based on the plurality of control signals, wherein the plurality of control signals represents a plurality of operating amplitude and frequency of the first bone-conducting vibrator and the second bone-conducting vibrator, wherein the wearable device operates the second bone-conducting vibrator through the vibrator adjustment unit;
a wearable device data transceiver, wherein the wearable device data transceiver is in data communication with the vibrator adjustment unit and the communication device, wherein the wearable device daft transceiver receives the plurality of control signals from the communication device;
a memory device storing the plurality of control signals representing a plurality of operating amplitude and frequency of the first bone-conducting vibrator and the second bone-conducting vibrator;
a data computing device being in data communication with the communication device and transmitting the plurality of control signals to the wearable device transceiver, the data computing device comprises:
a data input unit receiving a plurality of data representing disease history;
a data computing device memory storing the plurality of control signals and coupled to the data input unit;
a data competing device transceiver coupled to the data computing device memory; and
a display coupled to the data computing device memory;
a server remote from the biomechanical wave control system and the data computing device, wherein the data computing device is in data communication with the biomechanical wave control system, wherein the data computing device transceiver transmits the plurality of data to the server and receives the plurality of control signals from the server, wherein the server transmits the plurality of control signals to the data computing device transceiver, the server comprises:
a data matching unit matching the plurality of data with the plurality of control signals;
a server central processing unit;
a server memory; and
a server transceiver.

2. The system for interactive intervention based on biomechanical wave as claimed in claim 1, wherein the first bone-conducting vibrator and the second bone-conducting vibrator are configured to output the plurality of control signals through vibration of biomechanical wave at the plurality of operating amplitude and frequency.

3. The system for interactive intervention based on biomechanical wave as claimed in claim 1, wherein the wearable device further comprises a back strap and a waist strap, wherein the back strap is coupled to at least one of the first bone-conducting vibrator and the second bone-conducting vibrator, the waist strap is coupled to at least one of the first bone-conducting vibrator and the second bone-conducting vibrator.

4. The system for interactive intervention based on biomechanical wave as claimed in claim 1, wherein the wearable device further comprises a power inlet port coupled to the control board.

5. The system for interactive intervention based on biomechanical wave as claimed in claim 1, wherein the data computing device is a computer.

6. The system for interactive intervention based on biomechanical wave as claimed in claim 1, wherein the server memory stores the plurality of control sign is configured to match various the plurality of data.

7. A method for interactive intervention based on biomechanical wave, comprising:
receiving, at a data computing device, a plurality of data representing disease history;
transmitting, from the data computing device, the plurality of data representing disease history to a server;
associating, at the server, the plurality of data representing disease history with a plurality of control signals;
transmitting, from the server, the plurality of control signals to a data computing device transceiver at the data computing device;
receiving the plurality of control signals at the data computing device transceiver;
transmitting, from the data computing device transceiver, the plurality of control signals to communication device of a biomechanical wave control system;
transmitting, from the communication device of the biomechanical wave control system to a wearable device data transceiver, the plurality of control signals;
receiving, at the wearable device data transceiver, the plurality of control signals;
storing, at a memory device, the plurality of control signals;
transmitting, from the wearable device data transceiver, the plurality of control signals to a vibrator adjustment unit;
converting, at the vibrator adjustment unit, the plurality of control signals into amplitude and frequency data;
transmitting, from the vibrator adjustment unit, the amplitude and frequency data to a first bone-conducting vibrator and a second bone-conducting vibrator;
receiving, at the first bone-conducting vibrator and the second bone-conducting vibrator, the amplitude and frequency data; and
operating the first bone-conducting vibrator and the second bone-conducting vibrator through vibration at operating amplitude and frequency corresponding to the amplitude and frequency data.

8. The method for interactive intervention based on biomechanical wave as claimed in claim 7, wherein the data computing device is a computer.

9. The method for interactive intervention based on biochemical wave as claimed in claim 7, wherein the first bone-conducting vibrator and the second bone-conducting vibrator is disposed within a back strap and a waist strap.

10. The method for interactive intervention based on biomechanical wave as claimed in claim 7, wherein the vibrator adjustment unit is capable of adjusting the operating amplitude and frequency through an amplifier adjustment controller.

11. The method for interactive intervention based on biomechanical wave as claimed in claim 7, wherein the wearable device further comprises a power inlet port coupled to the first bone-conducting vibrator and the second bone-conducting vibrator.

12. The method for interactive intervention based on biomechanical wave as claimed in claim 11, where in the power inlet port receives electric power supply to power the biomechanical wave control system.
